(19) United States
(12) Patent Application Publication
(10) Pub. No.: US 2011/0295123 A1
(43) Pub. Date: Dec. 1, 2011

(54) CURVED LINEAR ARRAY TRANSDUCER, SYSTEM AND METHOD

(75) Inventor: Ernest J. Feleppa, Rye, NY (US)

(73) Assignee: RIVERSIDE RESEARCH INSTITUTE, New York, NY (US)

(21) Appl. No.: 12/791,051

(22) Filed: Jun. 1, 2010

(51) Int. Cl. A61B 8/14

(52) U.S. Cl. 600/447

(57) ABSTRACT

A focused ultrasound beam is created using a probe having a curved linear array. The curved linear array is asymmetric and two dimensional comprising individual parallel circumferential, linear sub-arrays disposed at a distal end of a probe. A first sub-array is disposed proximate a distal end and a final sub-array is disposed proximate a proximal end with subsequent sub-arrays disposed between the first and final array. In practice, the first sub-array transmits a set of pulses of ultrasound radiation for a period of time then an adjacent array transmits a set of pulses of ultrasound radiation after a lag. The transmissions by each sub-array continue with a lag between each array transmission until the final sub-array transmits a final set of pulses of ultrasound radiation. The pulse is generated by each transmission is steered and focused depending on the phases and amplitudes of the pulses of ultrasound radiation transmitted by the full set of elements in the transmitting sub-arrays. The set of multiple pulses permits interrogating individual voxels in a scanned volume from different directions.

### PRIORITY AND RELATED APPLICATION

N/a

### FIELD OF THE INVENTION

The present invention relates to ultrasonic transducers, more particularly, the present invention relates to an array employed for sector-scan applications.

### BACKGROUND OF THE INVENTION

One of the several presently available noninvasive techniques for medical diagnosis includes the use of ultrasound to produce ultrasonic images of portions of the human body which would otherwise be inaccessible except by surgery. Ultrasound devices generally require the use of probes which can be applied either externally or internally with respect to the body in order to produce the appropriate image. External applications access the body transcutaneously. Internal applications provide access to the body through body cavities such as the esophagus, vagina, and rectum, through a blood vessel, through laparoscopic surgery, or through open surgery. Probes that access the body through body cavities are termed intracavity probes; probes that access the body through blood vessels are termed intravascular probes.

An ultrasonic scanning probe samples echo-signal data so that an image can be made of a cross-sectional slice or plane through the body. Known intravascular or intracavity probes typically are cylindrical and include either a linear ultrasonic transducer array that extends along the longitudinal axis of the probe or a curved circumferential linear array that extends either completely around or partially around the body of the probe. In either case, the imaging elements typically are capable of providing only one type of planar cross-sectional view of the tissue or other structures surrounding the probe. Prior devices containing a linear array parallel to the probe axis produce an image representing a slice along the length of the probe; prior devices containing a circumferential or partially circumferential array produce an image representing a slice transverse to the length of the probe. Imaging is accomplished by causing an ultrasonic beam to scan back and forth in the plane of the image. Earlier devices also are able to capture an image by use of a moving single-element ultrasonic transducer located at the tip of the probe that scans either a longitudinal or transverse plane. Additionally, biplanar devices include both a linear and circumferential ultrasonic transducer array for capturing an image that is parallel to the probe axis and also an image that is transverse to the probe axis. Earlier devices that incorporate single-element transducers also can scan in longitudinal and transverse planes either by using two separate single-element transducers or by rotating the scanning plane of one single-element transducer.

In ultrasound imaging, a human body is exposed to brief ultrasonic pulses with ultrasound echo signals being recorded and displayed. To send and receive the ultrasound pulses according to such a pulse-echo method, modern probes use piezoelectric transducer elements arranged in an array. These transducer elements can be arranged in a straight linear (one-dimensional) row or chain (a so-called linear array) and are controlled by an electronic control unit, separately or in groups, to achieve a directing effect. A linear array can be flat and can be oriented on the flat face of a probe, or in the present application, with its long axis and therefore with its scanning plane parallel to the axis of a cylindrical probe or it can be curved to wrap around a cylindrical probe either partially or completely and therefore with its scanning plane perpendicular to the probe axis. The directional control of the ultrasound beam takes place by time-delayed transmission of the individual elements in the transmission case, where the desired beam direction results from superimposition of the waves proceeding from the elements, pursuant to Huygens' principle. In the reception case, the desired angle-dependent sensitivity is also achieved by time-dependent or phase-dependent superimposition of the time signal progressions recorded by the individual elements. Arrays of ultrasound transducer elements controlled in this manner are therefore also referred to as “phased arrays.” Using such phase-delayed controlled linear arrays, ultrasound beams can be focused in a plane formed by the transducer elements on the array surface.

U.S. Patent Publication No. 2005/0124884 discloses multidimensional transducer systems and methods for intra patient probes. A matrix arrangement of electrodes and associated connections with an imaging system are provided. This transducer uses these intersecting electrodes to select active elements by using a small number of leads. Different planes are rapidly imaged by electronically switching the selected aperture. U.S. Patent Publication No. 2009/0030317 discloses ultrasonic imaging devices, systems, and methods that includes one or more channels for delivering ultrasound pulses.

What is needed is an ultrasound probe that has parallel circumferential transducer arrays that can be phased with respect to each other. It is desired that sector-scan planes scanned by the ultrasound probe be capable of being directed at a particular angle with respect to the probe axis. It is further desired that the probe be able to interrogate voxels in a scanned volume from different directions, which could also reduce speckle. Scanning a given voxel from different directions with non-coherent superposition of the resulting signals is termed spatial compounding.

### BRIEF SUMMARY OF THE INVENTION

A focused ultrasound beam is created by providing a probe having a curved linear array. The curved linear array comprises a closely spaced set of individual curved parallel linear sub-arrays disposed at a distal end of a probe around a probe axis. The curved linear array is asymmetric and two dimensional. Each individual sub-array includes finely spaced circumferential elements that permit focusing and beam forming scan vectors that cover a sector angle. A first sub-array is disposed proximate to a distal end and a final array is disposed proximate to a proximal end with subsequent sub-arrays disposed between the first and final sub-array. In practice, the first sub-array transmits radiation for a predetermined period of time followed by a transmission from an adjacent sub-array after a predetermined time delay. The transmission of subsequent sub-arrays continues with a delay between each sub-array transmission until the final sub-array transmits radiation. The phasing of the delays among the set of sub-arrays determines the position of the scanned plane with respect to the probe. The location and angle of the plane with respect to the probe axis define the orientation of the scan plane. While this description applies to transverse scan planes, a similar control of scan-plane orientation can be applied to longitudinal scan planes. For either longitudinal or transverse scan planes, the combined transmissions generate a directed wave that permits interrogating voxels in a scanned volume from different directions.

### BRIEF DESCRIPTION OF THE DRAWINGS

FIG. 1 is schematic illustration of an ultrasonic imaging system of the present invention.

FIG. 2 is schematic view of a distal portion of a probe according to the present invention.

### DETAILED DESCRIPTION OF THE INVENTION

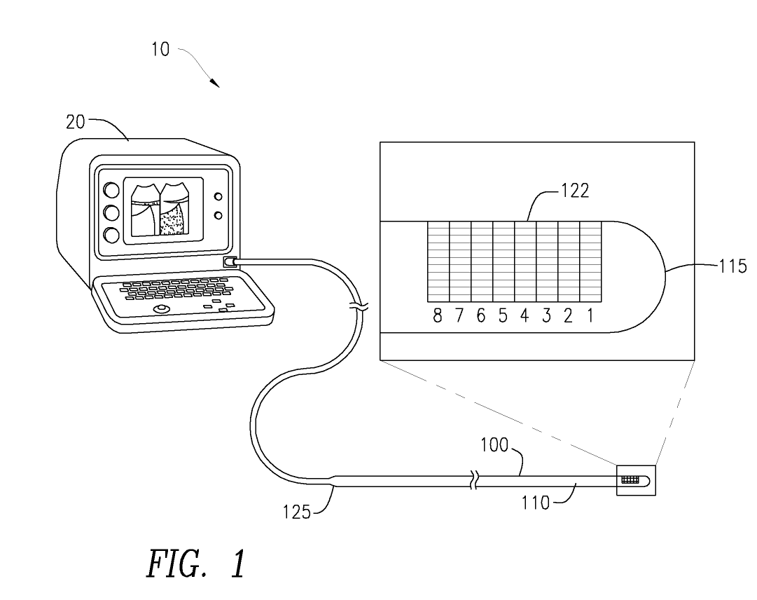

Referring to FIG. 1, there is shown a view of an ultrasonic imaging system 10 comprising an ultrasound scanner 20 that contains a suitable processor for controlling transmitted signals and processing returned echo signals. Scanner 20 is connected to an ultrasound probe 100 for insertion into the body of a patient. The probe 100 has no moving parts. The probe 100 has a body 110 with a distal end 115 and a proximate end 125. A curved linear array 122 is disposed on a distal end 115 of the body 110. The scanner 20 comprises a processor having suitable signal processing capability and a user friendly interface. The scanner 20 controls the phasing and amplitudes of transmitted signals from every element in every sub-array in the probe and processes signals detected at every element of every sub-array in the probe 100 and displays images that are derived from the acquired signals. The scanner 20 enables directional transmitted-pulse sequencing and echo-signal reception as will be discussed infra.

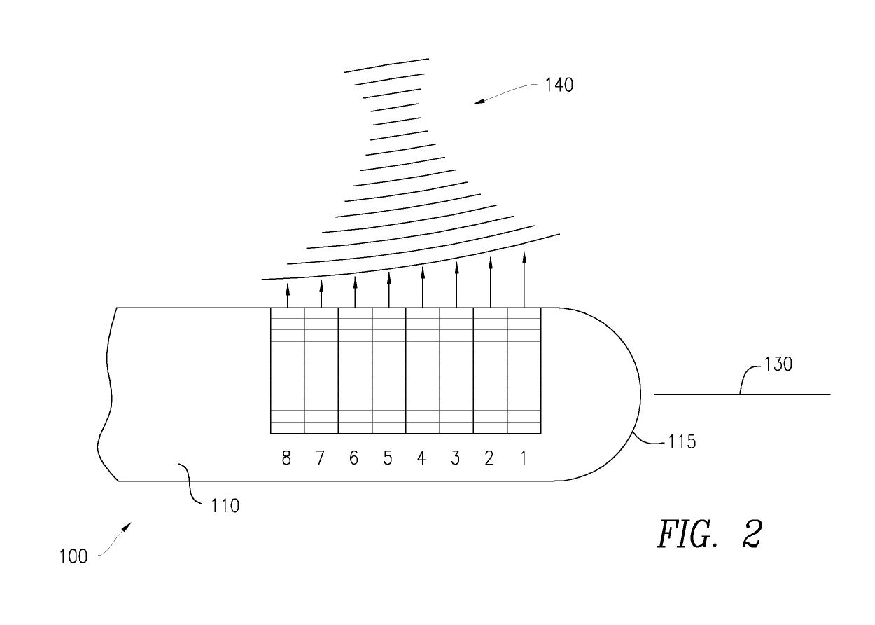

In one embodiment, the curved linear array 122 covers at least about one hundred-twenty degrees (120°) circumferentially about the probe axis 130 (FIG. 2.) The array 122 is asymmetric and two-dimensional and comprises a series of uniform and closely spaced arrays. In one embodiment, there would be 40 side-by-side circumferential arrays disposed on the distal end of the probe body 110. In the embodiment shown in the figures, the probe 100 has eight parallel, circumferential array rows (1-8). The first array row is proximate to the distal end 115 of the probe 100 and the final or eighth row 8 is closest to the proximate end 125 of the probe 100. The individual arrays 1-8 are situated next to each other for some distance along the length of the probe 100. The parallel orientation of each array (1-8) in the arrays 122 permits a set of sector scans to be obtained over a full volume of the body target, such as a prostate gland in an intracavity examination or such as arterial plaque in an intravascular examination.

Each individual array (1-8) of the curved linear array 122 is comprised of finely spaced elements for focusing and beam forming scan vectors. The number of elements in the curved linear sub-array can vary and the set of sub-arrays can cover any required length along the linear probe-axis direction. It is to be understood that any number of elements can be used depending on the particular application. The elements in one embodiment are formed of ceramic piezoelectric material, such as, for example, lead zirconate titanate (PZT). However, other piezoelectric materials can be employed.

The parallel configuration of the arrays 120 permits phasing of transmitted radiation between individual, adjacent arrays. This phasing creates a wave in a sector-scan plane that can be directed at a particular angle with respect to the probe axis 130. In one method of the present embodiment, a time delay is present between pulses emitted by each of the individual sub-arrays 1-8 of the curved linear array 122. The time delay is brief compared to the period (1/frequency) of the subsequent radiation pulses.

As one example, FIG. 2 shows the sequence of transmission being from sub-array 1 to sub-array 8. The first sub-array 1 is excited for a time period, then after a time delay sub-array 2 is excited. The delay between the transmission of the first sub-array 1 and the transmission of the sub-array 2 is shorter than the period of said radiation for the first sub-array. The subsequent sub-arrays 3-7 are likewise individually excited with a delay between each sub-array until the final sub-array 8 is excited. Here too, the delay between the transmission of the sub-array 2 and the transmission of the next subsequent sub-array 3 is shorter than the period of said radiation for the sub-array 2 and so on.

The individual transmission from each array 1-8 in the curved linear array 120 generates a focused wave 140. The focused wave 140 is directed to the left (from array 1 to array 8) as the pulse from each earlier transmitting array generates an interference pattern with each later-transmitted array in accordance with Huygen's Principal. The phasing or pulsing sequence of the method of the present invention causes the focused wave 140 to be directed in any intended direction. If the pulsing sequence were reversed with pulses emitting initially with individual sub-array 8 and ending with sub-array 1, then the focused wave 140 will be directed in the opposite direction. The direction of plane of the focused wave 140 may be altered based on the selection order and exact time delays of transmission from each sub-array in the curved linear array 120. The axis of the transmitted focused beam may be moved around the probe by selecting and phasing elements within each sub-array. Also not all sub-array elements fire with each pulse and those that do fire may not fire with the same amplitudes, i.e. apodizations can be applied over the circumferential elements in a sub-array.

By phasing the emission of the curved linear array 122 of the present invention the probe 100 may remain stationary when in use. The curved linear array 122 is further advantageous in that the parallel circumferential sub-arrays can be phased with respect to each other so that the sector-scan plans can be directed in any direction with respect to the probe axis. The directed wave 140 permits interrogating voxels in a scanned volume from different directions, which consequently reduces speckle.

While the invention has been described by way of example and in terms of specific embodiments it is not so limited and is intended to cover various modifications as would be apparent to those skilled in this art area. Therefore, the scope of the appended claims should be accorded the broadest interpretation so as to encompass all such modifications.

1. A method of obtaining ultrasound sector scans of a tissue comprising:
providing a probe having a plurality of circumferential curved ultrasound sub-arrays, each sub-array having a plurality of component array elements;
transmitting an initial pulse of ultrasound radiation from a first pair of a selected subset of elements in a sub-array among said plurality sub-arrays;
transmitting a pulse of ultrasound radiation from at least one subsequent pair of sub-array elements of said plurality of sub-array elements said subsequent transmitted pulse not necessarily having the same pulse amplitude as that of the initial transmitted pulse to allow for apodization;
transmitting sequential individual pulses of ultrasound radiation from all remaining pairs of sub-array elements of said plurality of sub-array elements until a preselected sub-array element transmits its pulse, each sequential pulse not necessarily having the same pulse amplitude as that of the preceding pulse to allow for apodization and
wherein a time delay between each ultrasound pulse transmitted from each pair of sub array elements can vary to provide control of the direction in which the ultrasonic pulse propagates, as long as the time delay is less than a period of said ultrasound being transmitted from the initial sub-array element.

2. The method of claim 1, wherein said array elements are asymmetric.

3. The method of claim 1, wherein said array elements are two-dimensional.

4. The method of claim 1, wherein said array elements comprise finely spaced elements, said finely spaced elements permitting focusing of scan vectors.

6. The method of claim 1, wherein said directed wave permits interrogating voxels in a scanned volume from different directions.

7. A method of directing an ultrasound beam comprising:
providing a probe having a plurality of circumferential curved ultrasound sub-arrays consisting of a plurality of component sub-array elements, said array elements being asymmetric;
transmitting an ultrasound pulse from a first sub-array element of said plurality of curved ultrasound array elements;
transmitting an ultrasound pulse radiation from a subsequent sub-array element of said plurality of curved ultrasound array elements; and
transmitting ultrasound radiation from a final sub-array element of said plurality of curved ultrasound array elements; and
wherein a time delay between the ultrasound pulse transmitted from the first pair of sub-array element and the ultrasound pulse transmitted from any subsequent sub-array elements can vary to provide control of the direction in which the ultrasound pulse propagates as long as the delay is less than a period of said ultrasound pulse being transmitted from the first sub-array element,

8. The method of claim 7, wherein said array elements are two-dimensional.

9. The method of claim 7, wherein said array elements comprise finely spaced elements, said finely spaced element permitting focusing of scan vectors.

10. The method of claim 7, wherein said array elements comprise finely spaced elements, said finely spaced element permitting beam forming scan vectors.

11. The method of claim 7, wherein said arrays are parallel circumferential arrays.

12. The method of claim 7, wherein said directed wave interrogates voxels in a scanned prostate volume from different directions.

13. The method of claim 12, wherein said directed wave obtains a set of sector scans.

14. A method for imaging prostate volume by phase sequencing adjacent arrays comprising providing a time delay between an ultrasound pulse transmitted from an initial array element and an ultrasound pulse transmitted from a subsequent array element, wherein the time delay is less than the period of said ultrasound pulse transmitted from the initial array.

15. The method of 16, wherein each said ultrasound pulse transmitted from the initial array and each ultrasound pulse transmitted from the subsequent array element creates a directed waveform, said directed wave form permitting voxel interrogation in a scanned prostate volume from different directions.
